(19) United States
(12) Patent Application Publication
(10) Pub. No.: US 2015/0164678 A1
(43) Pub. Date: Jun. 18, 2015

(54) VACUUM SUCTION APPARATUS

(71) Applicants: Chung-Liang Lee, Nantou County (TW); Po-Cheng Li, Taichung City (TW)

(72) Inventors: Chung-Liang Lee, Nantou County (TW); Po-Cheng Li, Taichung City (TW)

(21) Appl. No.: 14/412,177

(22) PCT Filed: Jan. 24, 2013
(86) PCT No.: PCT/CN2013/070909

§ 371 Date: Dec. 30, 2014

(51) Int. Cl. A61F 5/41

(52) U.S. Cl. CPC A61F 5/41; A61F 2005/412

(57) ABSTRACT

A vacuum suction apparatus includes a cup body, an adjust member, and a seal assembly. Therein, the cup body is hollowed with two penetrable ends, having a neck portion disposed with penetrable first and second openings at both ends thereof, while the first opening is combined with a joining member. The adjust member is screwed to the joining member. The seal assembly has an upper partition, a lower partition, and a membrane, cooperating with the adjust member to allow the air to pass through the upper partition, the lower partition, and the adjust member. A negative pressure is produced due to the seal function of the membrane, such that a male genital organ reciprocates to feel the force of withdrawing and pushing. The forces are adjustable with the adjust member, and the user is able to control the speed of the apparatus reciprocation.

### BACKGROUND OF THE INVENTION

1. Field of the Invention

The present invention relates to vacuum suction apparatus, more particularly, to a vacuum suction apparatus for aiding male genital organ erection.

2. Description of the Related Art

Referring to FIG. 5, a prior art of a vacuum suction assisting device 91 for aiding male erection comprises a cup body 910 combined to a push type suction member 911 by a tube 912. During operation, a male genital organ is sleeved by the cup body 910, while a negative pressure is produced when a ball member 913 of the suction member 911 is pushed, thereby aiding the erection of the male genital organ.

Said vacuum suction assisting device is operated with the external component operation producing a negative pressure in the cup body 910; however, when the operation of the external component is ceased, the negative pressure in the cup body thus disappears. User needs to maintain the operation of the external component. Referring to said vacuum suction assisting device 91, continuous suction is laborious. Therefore, such a vacuum suction assisting device operated through external components needs to be improved.

Thus, the present invention aims at solving the issues during the operation of such vacuum suction assisting devices.

### SUMMARY OF THE INVENTION

The objective of the present invention is to solve the abovementioned issues by providing a vacuum suction apparatus, wherein the air inflow and air outflow are adjustable according to the user's sensation, thereby adjusting the negative pressure during the male genital organ being withdrawn from the cup body and the resistance during the male genital organ being inserted into the cup body. The present invention facilitates the erection of male genital organ and also serves as a sex toy.

For achieving the abovementioned objectives, the present invention provides following technical solutions:

a vacuum suction apparatus, comprising:

a cup body, hollowed with two penetrable ends, having an outer diameter tapering at one end to form a neck portion, a penetrable first opening disposed in the neck portion, and a penetrable second opening disposed on the other end of the neck portion, while the first opening is combined with a joining member;

an adjust member, screwed to the joining member with an air permeable gap therebetween, which is capable of being adjusted between different sizes by the adjust member rotating against the joining member, while the joining member is provided with at least a longitudinal air channel for facilitating the air flow between the adjust member and the joining member; and

a seal assembly, including a stop member, a membrane seat, and a membrane, while the stop member has an upper partition with an upper air bore; the membrane seat is provided with a lower partition disposed beneath the upper partition and having a lower air bore, and a membrane chamber is formed between the upper partition and the lower partition; the membrane is disposed in the membrane chamber and provided with a slit, and an air passable path is formed by the upper air bore, the slit, and the lower air bore.

The cup body is internally provided with a hollow rubber sleeve, such that the rubber sleeve is placed in the cup body. Also, the rubber sleeve further has an input end having a tapering bore diameter. A partition membrane is disposed in the rubber sleeve.

The upper partition is provided with an upper concave at the position of the upper air bore. The membrane seat is provided with a lower concave at the position of the lower air bore. A movement space is formed of the lower concave and the upper concave. The lower air bore of the lower partition is smaller than the upper air bore of the upper partition.

The seal assembly further comprises a jacket and a jacket membrane, while the jacket membrane mounts around the bottom of the jacket, thereby being combined to the bottom of the membrane seat by use of the jacket.

The cup body is provided with a stretch sleeve on the end thereof adjacent to the second opening.

The membrane has plural upper and lower convex dots correspondingly disposed on the top and bottom surfaces thereof, wherein the lower convex dots are smaller than the upper convex dots.

As for the advantages provided by the present invention, the present invention, based on the user's sensation, is able to adjust the air inflow and outflow of the cup body during operation of the present invention, such that the negative pressure produced during the male genital organ withdrawn and the resistance produced during the male genital organ inserted into the cup body are adjusted, so as to facilitate the erection of male genital organ and at the same time serve as a sex toy.

The abovementioned and other advantages of the present invention are able to be clearly understood by referring to following detail description and drawings.

Alternative embodiments of the present invention may have different configurations. The embodiment herein provided is explained in detail in the specification of the present invention, and the structures thereof are illustrated in the drawings appended.

### BRIEF DESCRIPTION OF THE DRAWINGS

FIG. 1 is a perspective view of a vacuum suction apparatus in accordance with the present invention.

FIG. 2 is a sectional view of the vacuum suction apparatus in accordance with the present invention.

FIG. 3 is an exploded view of the vacuum suction apparatus in accordance with the present invention.

FIG. 4 is a schematic view illustrating the status of the vacuum suction apparatus during the male genital organ inserted and withdrawn therein.

FIG. 5 is a perspective view of a known vacuum suction assisting device.

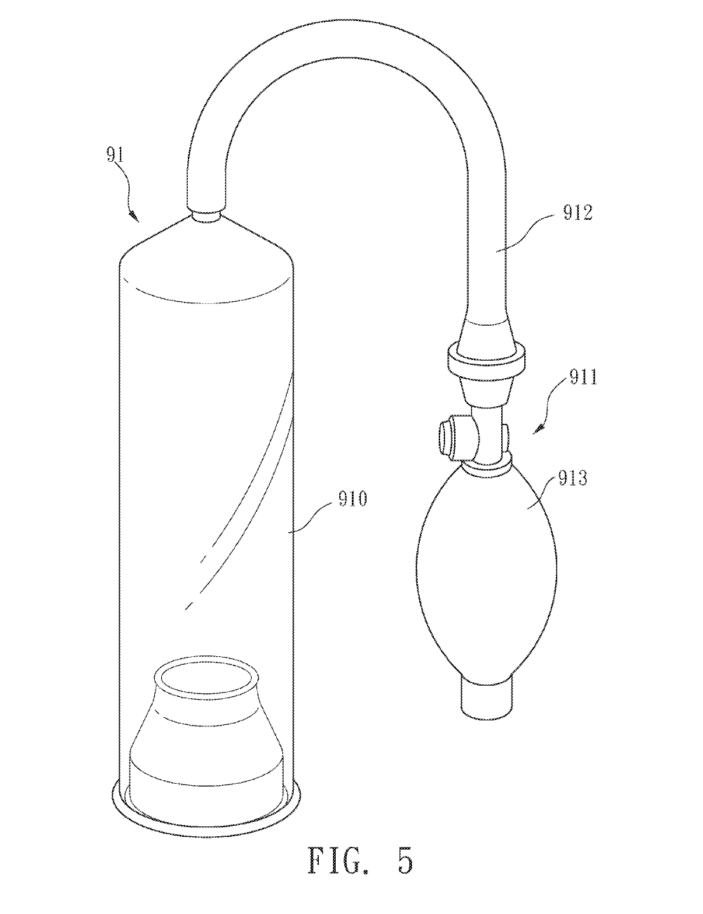

### DETAILED DESCRIPTION OF THE INVENTION

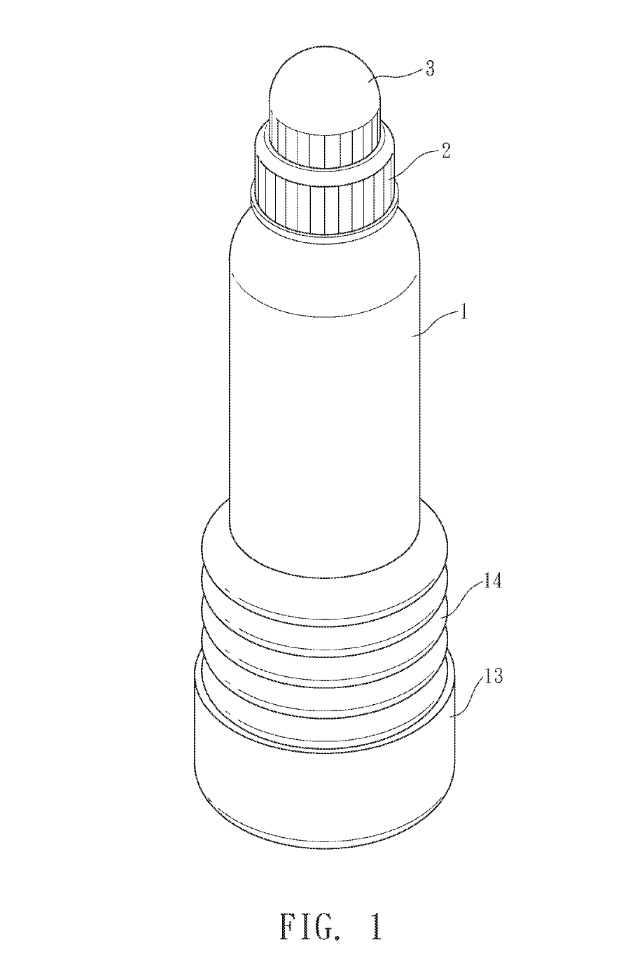

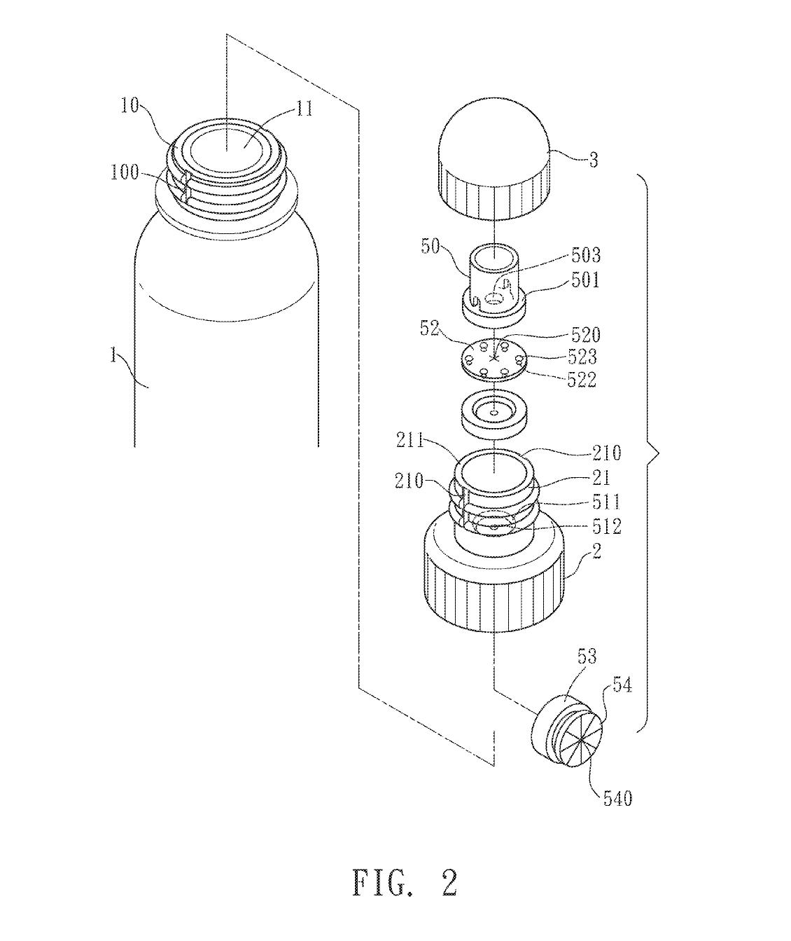

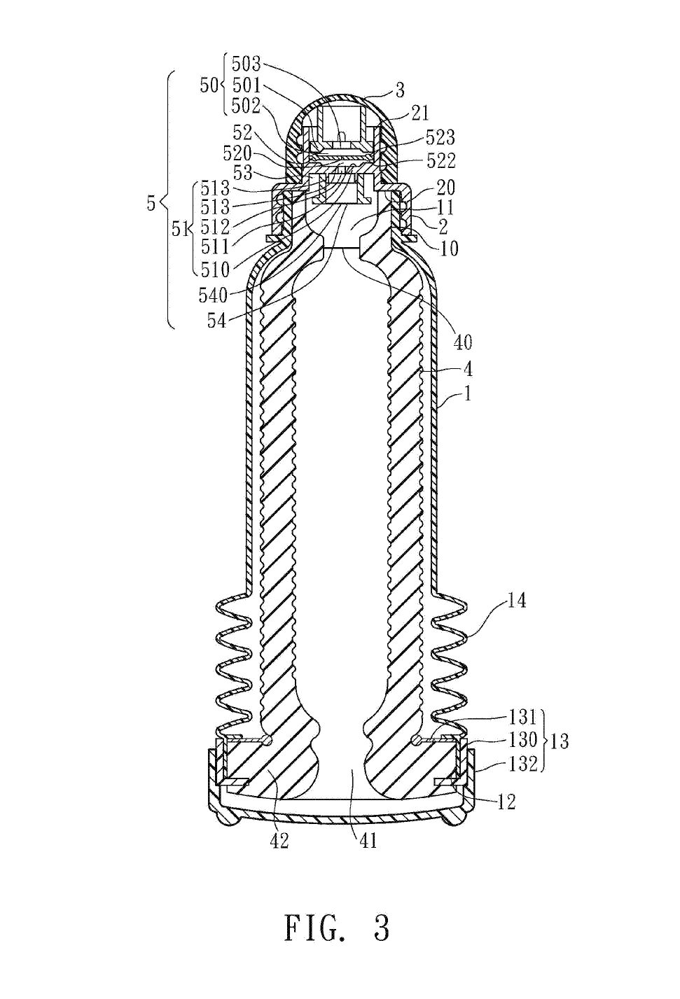

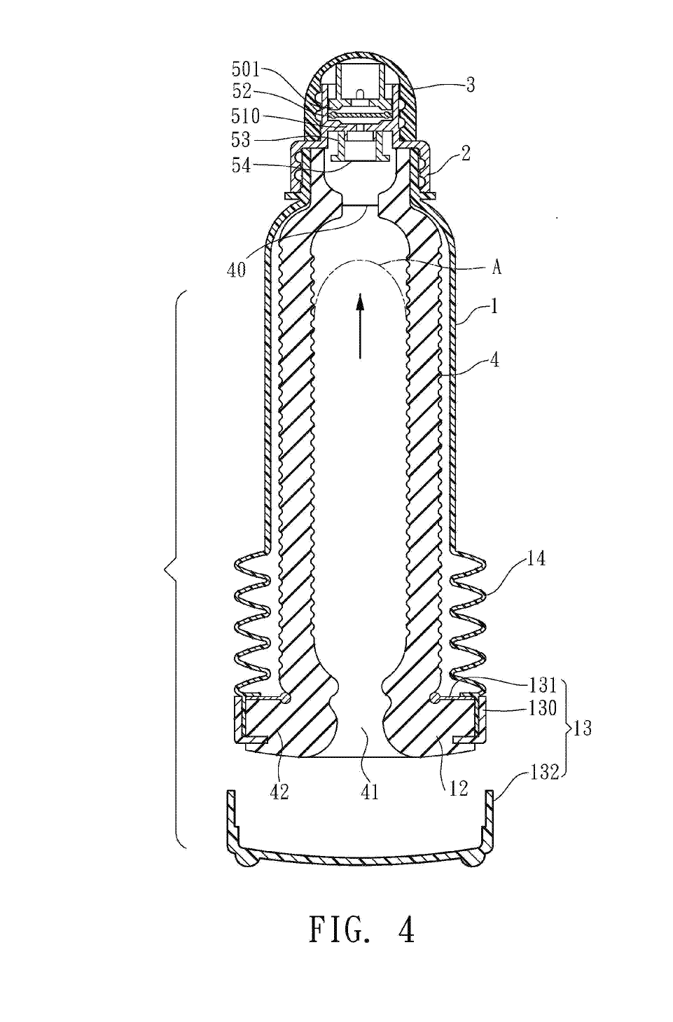

A vacuum suction apparatus is provided by the present invention. Referring to FIG. 1 to FIG. 4, a total of five embodiment structures are applied only for illustrating the present invention, with no intension to limit the scope of the invention. Each embodiment is illustrated as following.

Referring to FIG. 1 to FIG. 4, where a first embodiment of the present invention is shown, the embodiment comprises a cup body 1, a joining member 2, an adjust member 3, a rubber sleeve 4, and a seal assembly 5.

Therein, the cup body 1 is hollowed with two penetrable ends and provided with a first opening 11. A neck portion 10 is disposed on the cup body 1 and provided with a penetrable second opening 12. Two ends of the cup body 1 are penetrable with the first opening 11 and the second opening 12 provided thereon, respectively, while the first opening 11 is combined with the joining member 2.

The cup body 1 of the embodiment has a seat 13 on one end of the second opening 12, wherein the seat 13 comprises an inner ring 130 integrally formed of the cup body 1, with a sleeve ring 131 disposed within the inner ring 130 and an outer cover 132 combined to the inner ring 130. The outer cover 132 covers the inner ring 130 and is coupled therewith. One stretch sleeve 14 is disposed on one end of the cup body 1 adjacent to the second opening 12.

The joining member 2 is screwed to the neck portion 10, thereby sealing the first opening 11. When the joining member 2 seals the first opening 11, air is prevented from flowing in or out from the first opening 11. In the embodiment, one stretch portion 21 extends outward from the joining member 2 to form a block portion 211. The stretch portion 21 is allowed to be screwed to the adjust member 3. An air permeable gap is defined between the adjust member 3 and the stretch portion 21. Two longitudinal air channels 210 are provided to the stretch portion 21, such that the air channels 210 facilitate the air flow between the adjust member 3 and the cup body 1. In case the adjust member 3 is tightly screwed by the stretch portion 21, the bottom of the adjust member 3 resist against the upper part of the block portion 211. Also, the adjust member 3 is allowed to be unscrewed and loosened in order to adjust the gap between the bottom of the adjust member 3 and the block portion 211.

The rubber sleeve 4 is hollowed and inserted into the cup body 1. In the embodiment, the rubber sleeve 4 is disposed in the cup body 1 and provided with an input end 41 having a tapering bore diameter. The bore diameter of the input end tapers against the hollowed part of the rubber sleeve 4 for simulating a female vaginal orifice, such that the male genital organ of the user is allowed to enter from the input end 41. The rubber sleeve 4, which is hollowed and inserted into the cup body 1, is provided with a partition membrane at one end adjacent to the first opening 11, wherein the partition membrane 40 is integrally form on the rubber sleeve 4. Therein, water is allowed to pass the partition membrane 40, such that the partition membrane 40 is able to prevent the semen from spilling out of the rubber sleeve 4.

A bottom part 42 is disposed at the input end 41 of the rubber sleeve 4 and confined between the ring sleeve 131 and the outer cover 132. The ring sleeve 131 is provided with at least one sleeve inner diameter, capable of adjusting the inner bore diameter of the input end 41. The ring sleeve 131 is able to temporarily prevent the blood of a male genital organ A from flowing back, thereby maintaining the erection duration of the male genital organ A. Also, by mounting around the root part of the male genital organ A with at least one sleeve inner diameter, different tightness demands of the user are met.

The seal assembly 5, which in the embodiment extends from the inner side of the stretch portion 21, comprises a stop member 50, a membrane seat 51, a membrane 52, a jacket 53, and a jacket membrane 54.

Therein, the stop member 50 is provided with an upper partition 501, the upper partition 501 has an upper concave 502 and an upper air bore 503.

The membrane seat 51, in the embodiment, is an integral component of the stretch portion 21 and made of a rigid material, wherein the membrane seat 51 has a lower partition 510 disposed underneath the upper partition 501. The lower partition 510 is provided with a lower concave 511 and a lower air bore 512. A membrane chamber 513 is disposed between the lower concave 511 and the upper concave 502. The lower air bore 512, in the embodiment, is smaller than the upper air bore 503.

The membrane 52 is disposed in the membrane chamber 513. The top surface and the bottom surface of the membrane 52 are provided with plural convex dots, respectively, and also provided with a slit 520, such that air is allowed to enter or be locked from either side thereof. When the adjust member 3 rotates, bottom surface of the stop member 50 thus arises or lowers correspondingly. The membrane 52 is able to arise freely. The membrane member 52 is made of a flexible material, and plural convex dots are disposed on the top surface and the bottom surface thereof, respectively, wherein the lower convex dots 522 are smaller and lower than the upper convex dots 523. When the membrane 52 is placed in the membrane seat 51, the smaller lower convex dots 522 of the membrane 52 defines a gap against the membrane seat 51 and is thereby presented in a floating-like status. When the adjust member 3 rotates, the bottom surface of the stop member 50 thus arises or lowers correspondingly, thereby optionally adjusting the inflow or out flow of air inside the cup body 1.

The membrane 52 of the present invention defines a gap by use of the convex dots thereon resisting against the stop member 50, such that when the adjust member 3 is adjusted to cause the stop member 50 to arise and lower, the membrane 52 correspondingly arises and lowers. As a result, the present invention is able to accurately control the inflow and outflow of the air, thereby altering the intensity of the negative pressure in the cup body 1.

The jacket membrane 54 is disposed on the bottom part of the jacket 53, wherein the jacket 53 is made of a flexible structure, so as to mount around the bottom of the membrane seat 51 and be placed with the joining member 2. The jacket membrane 54 is provided with a slit 540, such that the jacket membrane 54 keeps the clearance of the membrane chamber 513, and at the same time allows the air in the cup body 1 to pass through the slit 540 of the jacket membrane 54.

During the operation of the vacuum suction apparatus of the embodiment provided by the present invention, male user holds the cup body 1 to mounts the cup body 1 around the male genital organ A, so as to operate the present invention in a manner of reciprocating thereon. Air in the cup body 1 passes from the first opening 11 through the jacket membrane 54 of the seal assembly 5, and then through the lower air bore 512 into the membrane chamber 513. The air pushes the membrane 52 into the upper concave 502, causing the membrane 52 to deform upward, thereby opening the slit 520 and enabling the air to pass the upper air bore 503 through the slit 520. The air in the cup body 1 also forms a resistance to the male genital organ A, while the air outflows from the middle of the joining member 2 and the neck portion 10. On the other hand, when the male genital organ A is withdrawn toward the input end 42, the air reversely flows back through the abovementioned structures into the cup body 1. When the air passes the membrane 52, the membrane 52 is pulled downward into the lower concave 511, causing the membrane 52 to deform downward, thereby opening the slit 520 and enabling the air to pass through the lower air bore 512 and the jacket membrane 54 into the cup body 1.

Thus, advantages of the present invention are obvious. The rotation of the adjust member 3 accurately adjust the gap between the adjust member 3 and the membrane seat 51 (as known as the stretch portion 21). With the membrane 52 allowing the air to pass through the slit 520, together with the difference between the upper convex dots 523 and the lower convex dots 522 of the membrane 52, the membrane 52 is thereby allowed to arise and lower for controlling the mutual airflow, such that when the male genital organ A is withdrawn in the cup body 1, the negative pressure therein produced an attraction to facilitate the erection of the male genital organ A. Furthermore, the negative pressure produced during the male genital organ A withdrawn and the resistance produced during the male genital organ A inserted to the cup body 1 simulates the reciprocation of the male genital organ A in a tight vagina. Therefore, the user is allowed to experience a virtual sexual intercourse.

In addition, during the user reciprocating the cup body 1, with the stretchable characteristic of the stretch sleeve 14, traveling distance of the male genital organ A in the cup body 1 is prolonged. Thus, the stretch sleeve 14 is applied to compress the air, so as to produce a negative pressure during the male genital organ A being withdrawn and inserted, thereby aiding the male genital organ A of the user capable of slightly erecting to be gradually sucked into the cup body 1 for carrying out the reciprocation.

Particular embodiments of the invention have been described in detail for purposes of illustration, so that various modifications and enhancements may be made without departing from the spirit and scope of the invention.

With the detailed explanation, skilled people in the field of the present invention are able to accomplish the present invention. Such contents meet the requirements of Patent Act, so that the present patent application is filed.

What is claimed is:

1. A vacuum suction apparatus, characterized in:
a cup body, hollowed with two penetrable ends, having an outer diameter tapering at one end to form a neck portion, a penetrable first opening disposed in the neck portion, and a penetrable second opening disposed on the other end of the neck portion, while the first opening is combined with a joining member;
an adjust member, screwed to the joining member with an air permeable gap therebetween which is capable of being adjusted between different sizes by the adjust member rotating against the joining member, while the joining member is provided with at least a longitudinal air channel for facilitating the air flow between the adjust member and the joining member; and
a seal assembly, including a stop member, a membrane seat, and a membrane, while the stop member has an upper partition with an upper air bore; the membrane seat is provided with a lower partition disposed beneath the upper partition and having a lower air bore, and a membrane chamber is formed between the upper partition and the lower partition; the membrane is disposed in the membrane chamber and provided with a slit, and an air passable path is formed by the upper air bore, the slit, and the lower air bore.

2. The vacuum suction apparatus of claim 1, characterized in that a hollow rubber sleeve is placed in the cup body and provided with an input end having a tapering bore diameter, while a partition membrane is disposed in the rubber sleeve

3. The vacuum suction apparatus of claim 1, characterized in that an upper concave is placed at the position of the upper air bore; the membrane seat is provided with a lower concave at the position of the lower air bore; a movement space is formed of the lower concave and the upper concave; the lower air bore of the lower partition is smaller than the upper air bore of the upper partition.

4. The vacuum suction apparatus of claim 1, characterized in that the seal assembly further comprises a jacket and a jacket membrane, while the jacket membrane mounts around the bottom of the jacket, thereby being combined to the bottom of the membrane seat by use of the jacket.

5. The vacuum suction apparatus of claim 1, characterized in that a stretch sleeve is provided on one end of the second opening adjacent to the second opening.

6. The vacuum suction apparatus of claim 1, characterized in that the top surface and the bottom surface of the membrane are provided with plural convex dots in a corresponding numbers, while the convex dots on the bottom surface thereof are smaller than the convex dots on the top surface thereof.
